(19) United States
(12) Patent Application Publication
(10) Pub. No.: US 2025/0205396 A1
(43) Pub. Date: Jun. 26, 2025

(54) APATHOGENIC COATING OF AN OBJECT, OBJECT HAVING THE APATHOGENIC COATING AND METHOD FOR ARRANGING AN APATHOGENIC COATING ON AN OBJECT

(71) Applicant: Siemens Aktiengesellschaft, München (DE)

(72) Inventors: Gabriele Winkler, Berlin (DE); Jens Dahl Jensen, Berlin (DE); Christian Doye, Berlin (DE); Ursus Krüger, Berlin (DE)

(21) Appl. No.: 18/850,315

(22) PCT Filed: Mar. 14, 2023
(86) PCT No.: PCT/EP2023/056434

§ 371 Date: Sep. 24, 2024

(30) Foreign Application Priority Data
Mar. 31, 2022 (EP) 22165865.1

(51) Int. Cl. A61L 31/08; A61L 31/16; C02F 1/46; C02F 1/461; C23C 14/16; C23C 14/30; C23C 14/58

(52) U.S. Cl. CPC A61L 31/088; A61L 31/16; C02F 1/4606; C02F 1/46176; C23C 14/16; C23C 14/30; C23C 14/5853; A61L 2300/102; A61L 2300/404; A61L 2300/606; A61L 2400/18; A61L 2420/02; C02F 2303/04

(57) ABSTRACT

An apathogenic (antimicrobial and/or antiviral) coating of an object surface of an object with at least one anode layer having at least one anode material and at least one cathode layer having at least one cathode material is provided, wherein the layers are designed such that a galvanic element is formed in the presence of moisture. Constituents of the pathogens are thus oxidatively destroyed. This inhibits the multiplication of pathogens on the object surface. Efficient disinfection of the object surface is possible. The aspect provided with the coating is in particular a medical implant, for example a stent.

### CROSS-REFERENCE TO RELATED APPLICATIONS

This application is a national stage of PCT Application No. PCT/EP2023/056434, having a filing date of Mar. 14, 2023, which claims priority to EP Application No. 22165865.1, having a filing date of Mar. 31, 2022, the entire contents both of which are hereby incorporated by reference.

### FIELD OF TECHNOLOGY

The following relates to an apathogenic coating of an object surface of an object, to an object having an apathogenic coating arranged on an object surface, and to a method of arranging the apathogenic coating on an object surface of the object.

### BACKGROUND

Apathogenic (antipathogenic or antimicrobial and/or antiviral) coatings for inhibition of the growth of pathogens on a surface of an object contain apathogenic substances such as metals or metal compounds (e.g., metal oxides). The apathogenic substances are added to paints, varnishes or polymer materials and then applied to the object surface of the object by painting or spraying.

Standard apathogenic substances are copper or copper compounds such as cuprite (copper oxide), copper thiocyanate, copper pyridine or silver (for example in the form of silver nanoparticles) or silver compounds such as silver chloride, silver nitrate, silver oxide, silver sodium hydrogen zirconium phosphate and silver zeolite A.

The metal ions (copper or silver ions) released from their metal compounds, and the silver nanoparticles, react with sulfur-and phosphate-containing enzymes in the cell membrane (cell wall) of a cell of the pathogen and hence disrupt a vital transport function of the cell membrane. The metal ions (for example essential calcium ions) are taken from the cell and bound to sulfur- and phosphate-containing macromolecules (for example to amino acids of proteins). The metal ions may also bind to a DNA (deoxyribonucleic acid) of a cell and hence prevent reproduction of the cell. The effects described lead to cell death.

However, copper compounds and silver compounds are not just toxic to microorganisms and/or viruses but can also be hazardous to higher life forms by the same mechanisms, especially in that they accumulate in the environment.

### SUMMARY

An aspect relates to an apathogenic coating of an object so as to avoid pollution of the environment (for example via the use of an object having the apathogenic coating or in the production of the object with the coating) with metal ions.

An aspect relates to an apathogenic coating of an object surface of an object having at least one anode layer having at least one anode material and at least one cathode layer having at least one cathode material, wherein the layers are configured such that a galvanic element is formed in the presence of moisture.

In a further aspect of embodiments of the invention, an object having an apathogenic coating arranged on an object surface of the object is specified, wherein the apathogenic coating at least inhibits replication of a pathogen on the object surface.

Finally, an aspect relates to a method of arranging the apathogenic coating on an object surface of an object, having the following method steps: a) providing the object having the object surface and b) arranging the coating on the object surface such that a galvanic element is formed in the presence of moisture.

The coating is apathogenic. Deposition or replication and hence accumulation of microbes (e.g., bacteria, fungi or algae) and/or of viruses on the object surface of the object is wholly or partly prevented. The coating thus has a disease-preventing effect without releasing materials that damage the environment (e.g., metal ions).

The galvanic element is an electrochemical reactor. The presence of moisture gives rise to an electrolyte required for the galvanic element. The layers form the electrodes of the galvanic element.

Moisture may be present in various media, for example in air (air humidity) or in perspiration (sweat), secretions and excrement of life forms. With the aid of moisture in the various media, the (micro) galvanic element is formed from the coating. This results in (micro) electrical fields. Reactive oxygen species such as hyperoxides (superoxides) and hydroxyl radicals are formed electrochemically at the (micro) cathode with the aid of oxygen dissolved in water. Nucleobases that are present in the nucleic acids DNA and RNA (ribonucleic acids) and are responsible for genetic information consist of a base skeleton of heterocyclic aromatic amines (purines and pyrimidines) having double bonds. The radicals mentioned have unpaired electrons in the outer electron shell and attack the double bonds of the amines in that they fill the outer electron shell thereof with the x electrons in order to achieve the noble gas configuration. This is associated with loss of the double bond system in the ring and the nucleobases are no longer able to pass on their information to protein biosynthesis, and the replication of the nucleic acids DNA and RNA is stopped. The microbes (microorganisms) are killed by oxidation.

For example, the microorganism also has a proteinogenic amino acid with sulfur. The sulfur in the amino acid can be oxidized to a sulfoxide by reactive oxygen species. A hydroxyl group in a side chain of a proteinogenic amino acid can be oxidized with the aid of reactive oxygen species to an aldehyde or carboxyl group. In any case, a chemically modified amino acid is formed. The chemically modified amino acid can no longer take part in the formation of vital proteins. Consequently, the microorganism will die.

In an embodiment, the anode material and/or the cathode material are porous. The electrodes of the galvanic element have pores. The pores are open. This increases a reactive surface area of the respective electrode. In addition, the electrolyte formed by moisture can be absorbed by the corresponding electrode.

It is particularly advantageous when the anode material has a redox potential (standard potential) of more than +1 V and the cathode material has a redox potential of below −1 V. These redox potentials are particularly suitable for initiation of electrochemical reactions for killing of pathogens and hence for efficient disinfection of the object surface of the object.

In embodiments, the anode material and/or the cathode material includes an elemental metal. In a particular configuration, the elemental metal is titanium. Titanium is especially used as anode material of the anode layer. Titanium is an example of a refractory metal (base, high-melting metal). Other examples of refractory metals are zirconium and hafnium (4^{th} transition group of the Periodic Table of the Elements), vanadium, niobium and tantalum (5^{th} transition group), and chromium, molybdenum and tungsten (6^{th} transition group).

In a particular configuration, the anode material and/or the cathode material include at least one metal compound. The metal compound is manganese dioxide (MnO₂). Manganese dioxide is especially used as cathode material in the cathode layer. It is particularly advantageous here to use porous manganese dioxide.

With regard to embodiments of the method arranging the coating on the object surface, preference is given to depositing the anode material and depositing the cathode material on the object surface. It is possible here for the layers to be applied indirectly or directly on the object surface. The layers would be applied indirectly, for example, even if they are arranged one on top of another on the object surface. If the layers are arranged alongside one another, the layers could also be applied directly to the object surface or multiple object surfaces.

The deposition of the anode material or the deposition of the cathode material thus gives rise to an apathogenic coating, the result of which is that at least one of the layers has at least one deposition.

Depending on the type of deposition, it is possible for any of the layers to have different layer thicknesses, for example in the um or in the nm range. In a particular configuration, at least one of the layers has a layer thickness selected from the range from 1 nm to 100 μm and especially from the range from 10 nm to 10 μm.

It is possible here to directly deposit the corresponding electrode material. It is alternatively conceivable to deposit not the electrode material but firstly a starting material (precursor) of the electrode material and then to convert the deposited starting material to the (actual) electrode material. In a particular configuration, therefore, the applying of the anode material and/or the applying of the cathode material comprise applying of at least one anodic starting material of the anode material and/or applying of at least one cathodic starting material of the cathode material.

In a particular configuration, a physical, chemical and/or physicochemical deposition method is employed for arrangement of the coating. The physical deposition method is, for example, a cathode ray atomization (sputtering), an electron beam evaporation or a cold gas spraying operation. In the case of very thin layers having layer thicknesses in the nm or in the sub-nm (atomic) range, a deposition method in the form of a chemical gas phase deposition or in the form of an atomic layer deposition may be employed.

For example, manganese dioxide can be applied to various materials (metals, ceramics, plastics) by numerous physical methods on the one hand and by chemical deposition on the other hand. Possible physical methods are reactive cathode ray atomization (reactive sputtering), electron beam evaporation of manganese the subsequent oxidation at 400 to 450° C. with dry air, and cold gas spraying.

The object is any article or any workpiece or any everyday article where pathogens can accumulate and replicate on one of the surfaces thereof. For example, the object is an article in everyday use, such as a banister or a grab rail in a public transport.

The object of the object surface of the object may consist of any object material. The object material is, for example, a metal, a ceramic or a plastic. Natural materials (e.g., stone) are also conceivable.

The aspect is a medical object. The medical object is especially a medical implant, a medical device, or an operating element for the medical device. The medical device is, for example, an operating instrument, an auxiliary of the operating instrument or a diagnostic system. Specifically, a medical device or the operating element of the medical device are generally used in a microbe-free or reduced-microbe environment in order to prevent replication of pathogens and hence infection of a patient.

The medical implant is an article which is inserted (implanted) within the patient's body and remains within the patient's body temporarily (over a prolonged period) or permanently. Examples of such medical implants are a heart pacemaker or an endoprosthesis (joint replacement), for example an artificial hip. In embodiments, the medical implant is a vessel prosthesis or a stent (medical implant for keeping vessels and cavities of the body open). The medical implant must also be sterile for insertion into the patient's body.

Embodiments of the invention obviate the need to disinfect the medical device, to disinfect the operating element of the medical device and/or to disinfect the medical implant. Sterility is assured.

The anode layer and the cathode layer may be arranged as desired on the object surface of the object. For instance, they may be arranged one on top of another or one alongside another. It is also possible for the layers to be in direct or indirect contact with the object surface.

In summary, embodiments of the invention is associated with the following advantages:
The apathogenic coating is very active and flexibly usable for a wide variety of different objects and applications.
The apathogenic coating does not release any toxic particles (ions, atoms or nanoparticles) to the environment.
The apathogenic coating is stable and does not undergo any chemical change. The coating acts like a catalyst that enables a chemical reaction in the first place or accelerates it without itself being consumed. The environment can thus be protected from toxic or corrosive chemicals that are used for decontamination of surfaces or disinfection of water or aqueous solutions (e.g., formaldehyde, phenols, hypochlorite, cuprite, copper pyridine or silver nanoparticle-containing substances).
It is possible to perform many catalysis cycles (especially in the case of use of titanium dioxide and manganese dioxide layers). Antimicrobial or antiviral active ingredients are not consumed and do not have to be replenished or replaced.
No waste products are formed.
Coating material is not consumed. There is no need for renewal or repeated application of the coating.
The use of disinfectants is likewise unnecessary.
To date, most medical implants such as stents have been manufactured from inert and corrosion-resistant alloys such as 316 L stainless steel, titanium alloys and Co-Cr alloys. In spite of the success of the use of stents in treatment of arterial occlusions, permanent presence in arterial vessels can lead to long-term complications such as thromboses or in-stent restenoses. New stents are produced from biodegradable metals such as iron, magnesium and zinc alloys. These temporary medical implants having a very thin titanium/manganese dioxide layer (layer thickness in the nano range) are protected from microbe attack. In the case of urinary stents, the adhesion of biofilms can be prevented by a thin Ti/MnO₂ coating.
In medicine, imaging devices such as computer tomographs, nuclear spin tomographs and x-ray instruments have to be calibrated. Phantoms are used for this purpose; these are filled with water. If the liquid is stored in the phantom for long periods, the liquid quality should remain the same. Colonization with microbes and microorganisms can be prevented by coating of the inside of the phantom with the apathogenic coating.
Operating elements of the medical devices and the surfaces thereof with which operating personnel and patients come into contact can likewise be provided with the apathogenic coating for freedom from microbes.
In public transport such as trains, the contact surfaces touched by passengers can also be protected from microbes by the apathogenic coating. This is especially also true of the sanitary area in trains.
In drive technology in the water economy, low-and high-pressure motors are used, which require an air/water heat exchanger or a water shell for cooling. The surfaces exposed to the cooling water can likewise be protected by embodiments of the invention from adhesion of microorganisms and formation of a biofilm.

### BRIEF DESCRIPTION

Some of the embodiments will be described in detail, with references to the following Figures, wherein like designations denote like members, wherein:

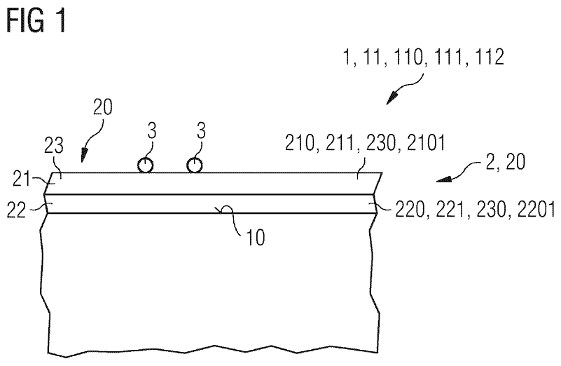

FIG. 1 shows a detail of an object having an apathogenic coating;

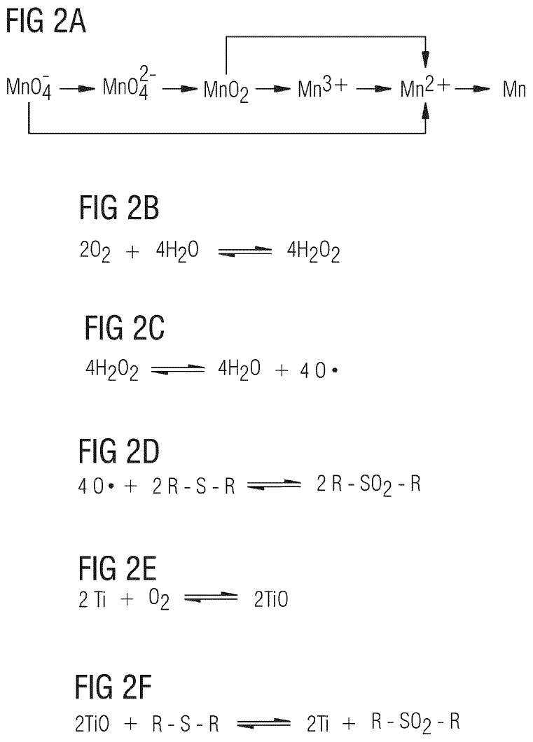

FIG. 2A shows various oxidation states of manganese and the into conversion thereof;

FIG. 2B shows a possible reaction at the microcathode;

FIG. 2C shows a possible reaction at the microcathode;

FIG. 2D shows a possible reaction at the microcathode;

FIG. 2E shows a possible reaction at the microanode;

FIG. 2F shows a possible reaction at the microanode;

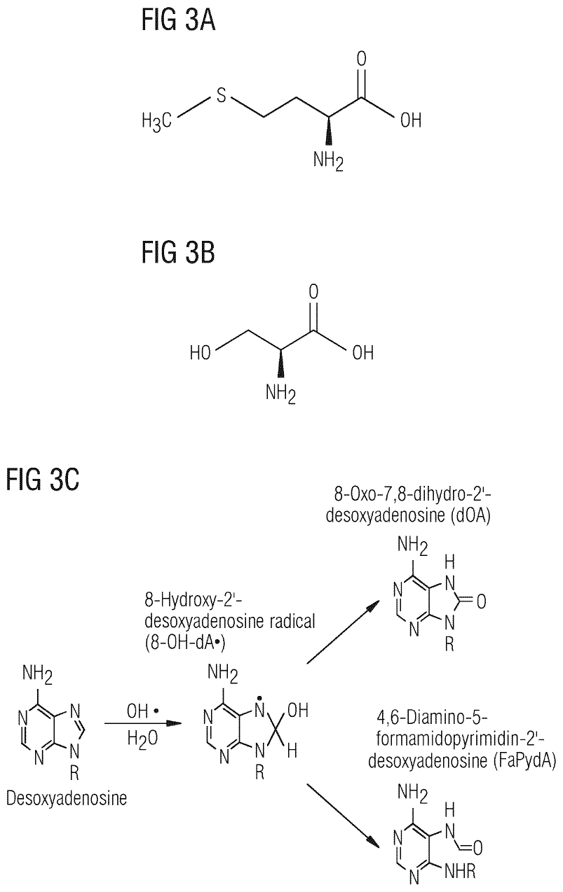

FIG. 3A shows the structural formula of methionine;

FIG. 3B shows the structural formula of serine; and

FIG. 3C shows the oxidation of adenine (nucleobases in DNA and RNA).

### DETAILED DESCRIPTION

In a first working example, an object 1 is given in the form of a stent (medical object 11, medical implant 110). In a further working example, the object 1 is the operating element 112 of a medical device 111.

On the object surface 10 of the object 1, an apathogenic coating 2 having an anode layer (microanode) 22 comprising elemental titanium 2201 as anode material 220 has been applied directly to the object surface 11.

The cathode layer (microcathode) 21 with cathode material 210 in the form of porous manganese dioxide (metal compound 2101) is present atop the anode layer 22. The layer thicknesses of the anode layer 21 and the cathode layer 22 are each about 100 nm. Both layers are gas phase depositions 230; for arrangement of the manganese dioxide layer 21, elemental manganese is first applied as cathodic starting material 211 by electron beam evaporation, and this is subsequently oxidized at 400 to 450° C.

Layers 21, 22 are configured such that a galvanic element (galvanic microelement) 20 is formed in the presence of moisture 23. This inhibits the replication of a pathogen 3 at the object surface 10.

Since manganese dioxide is nobler than titanium in terms of electrochemical standard potential, an electrical field is formed between the metal dioxide, manganese dioxide, and the metal, titanium, in the presence of moisture. This means that redox processes can proceed at the manganese dioxide and the titanium, and the electron transitions that take place can kill microbes.

The procedure for arrangement of the apathogenic coating 2 on the object surface 10 is as follows:
a) providing the object 1 having the object surface 10 and
b) arranging the coating 2 on the object surface 10 such that a galvanic element 20 is formed in the presence of moisture 23.

At the microcathode, the following reactions can be identified with the respective standard potentials (cf. FIG. 2A):

MnO₄⁻→MnO₄²⁻: 0.56 V

MnO₄⁻→Mn²⁺: 1.51 V

MnO₄²⁻→MnO₂: 2.09 V

MnO₂→Mn³⁺: 0.95 V

Mn³⁺→Mn²⁺: 1.54 V

MnO₂→Mn²⁺: 1.23 V

Mn^{2+→Mn: -}1.185 V

At the microcathode 21, reactions take place according to FIGS. 2B, 2C and 2D. At the microanode 22, the reactions according to FIGS. 2E and 2F occur.

FIGS. 3A and 3B (methionine and serine) are examples of proteinogenic amino acids, the functional groups of which can be oxidatively modified with the aid of embodiments of the invention.

FIG. 3C shows, by the oxidation of adenine (nucleobases in DNA and RNA), fundamental reactions that are triggered with the aid of embodiments of the invention in molecules of a pathogen, such that replication of the pathogen is impossible.

Although the present invention has been disclosed in the form of embodiments and variations thereon, it will be understood that numerous additional modifications and variations could be made thereto without departing from the scope of the invention.

For the sake of clarity, it is to be understood that the use of “a” or “an” throughout this application does not exclude a plurality, and “comprising” does not exclude other steps or elements.

1. An apathogenic coating of an object surface of an object comprising:
at least one anode layer having at least one anode material; and
at least one cathode layer having at least one cathode material;
wherein the at least one anode layer and the at least one cathode layer are configured such that a galvanic element is formed in a presence of moisture.

2. The apathogenic coating as claimed in claim 1, wherein the at least one anode material and/or the at least one cathode material are porous.

3. The apathogenic coating as claimed in claim 1, wherein the at least one anode material has a redox potential of above +1 V and the at least one cathode material has a redox potential of below −1 V.

4. The apathogenic coating as claimed in claim 1, wherein the at least one anode material and/or the at least one cathode material include an elemental metal.

5. The apathogenic coating as claimed in claim 4, wherein the elemental metal is titanium.

6. The apathogenic coating as claimed in claim 1, wherein the at least one anode material and/or the at least one cathode material include at least one metal compound.

7. The apathogenic coating as claimed in claim 6, wherein the at least one metal compound is manganese dioxide.

8. The apathogenic coating as claimed in claim 1, wherein at least one of the at least one layer and the at least one cathode layer has at least one deposition.

9. The apathogenic coating as claimed in claim 1, wherein at least one of the at least one anode layer and the at least one cathode layer has a layer thickness selected from a range from 1 nm to 100 μm.

10. An object having an apathogenic coating as claimed in claim 1 arranged on an object surface of the object, wherein the apathogenic coating at least inhibits replication of a pathogen on the object surface.

11. The object as claimed in claim 10, wherein the object is a medical object.

12. The object as claimed in claim 11, wherein the medical object is a medical implant, a medical device or an operating element for the medical device.

13. The object as claimed in claim 12, wherein the medical implant is a vessel prosthesis or a stent.

14. A method of arranging an apathogenic coating on an object surface of an object as claimed in claim 10, the method comprising:
a) providing the object having the object surface; and
b) arranging the apathogenic coating on the object surface such that a galvanic element is formed in a presence of moisture.

15. The method as claimed in claim 14, wherein the arranging of the apathogenic coating on the object surface comprises applying the anode material and applying the cathode material on the object surface.

16. The method as claimed in claim 15, wherein the applying of the anode material comprises applying at least one anodic starting material of the anode material and/or the applying of the cathode material comprises the applying of at least one cathodic starting material of the cathode material.

17. The method as claimed in claim 14, wherein the coating is arranged by employing a physical, chemical and/or physicochemical deposition method.

18. The apathogenic coating as claimed in claim 1, wherein at least one of the at least one anode layer and the at least one cathode layer has a layer thickness selected from a range from 10 mm to 10 μm.
